(19) United States
(12) Patent Application Publication
(10) Pub. No.: US 2026/0298950 A1
(43) Pub. Date: Oct. 1, 2026

(54) MICRO-FLUIDIC CHIP FOR BLOOD DETECTION AND DETECTION METHOD THEREOF

(71) Applicant: JIANGSU ZEA BIOTECHNOLOGY CO., LTD, Taizhou (CN)

(72) Inventors: Yuze WANG, Taizhou (CN); Jingbo CHEN, Taizhou (CN)

(73) Assignee: JIANGSU ZEA BIOTECHNOLOGY CO., LTD, Taizhou (CN)

(21) Appl. No.: 19/477,821

(22) PCT Filed: Jun. 6, 2023
(86) PCT No.: PCT/CN2023/098521

§ 371 Date: Oct. 23, 2025

(30) Foreign Application Priority Data
May 15, 2023 (CN) 202310541260.9

(51) Int. Cl. G01N 33/80; G01N 33/543

(52) U.S. Cl. CPC G01N 33/80; G01N 33/54313; G01N 33/54386

(57) ABSTRACT

A micro-fluidic chip comprises a chip body, the chip body comprises more than one separation and detection unit, each separation and detection unit comprises a trace blood separation tank and more than one reaction test unit, and each reaction test unit comprises a first sample loading chamber, a first L-shaped micro-channel, a second sample loading chamber, a second L-shaped micro-channel, a Y-shaped micro-channel and a reaction detection cavity; the Y-shaped micro-channel comprises a first sample injection port, a second sample injection port and an outflow port, and fluids respectively flowing into the first sample injection port and the second sample injection port are mixed and then discharged from the outflow port; the first L-shaped micro-channel communicates the first sample loading chamber; the second L-shaped micro-channel communicates the second sample loading chamber; and the outflow port of the Y-shaped micro-channel is communicated with the reaction detection cavity.

### CROSS-REFERENCE TO RELATED APPLICATIONS

This application is the U.S. national phase of International Application No. PCT/CN2023/098521 filed on 6 Jun. 2023 which designated the U.S. and claims priority to Chinese Application No. CN 202310541260.9 filed on 15 May 2023, the entire contents of each of which are hereby incorporated by reference.

### TECHNICAL FIELD

The present invention relates to a blood detection technology, and particularly to a micro-fluidic chip for blood detection and a detection method thereof.

### BACKGROUND

An ABO blood group system is a blood group system with a strongest antigen immunity in a human blood group system. A erythrocyte ABO blood grouping test is divided into a forward typing test and a reverse typing test according to serology. The reverse typing test is to detect an anti-A antibody and an anti-B antibody in serum with A and B reagent erythrocytes, which complements the forward typing test, thereby improving the accuracy of ABO blood grouping. Commonly used methods comprise a slide method, a test tube method, a microplate method and a micro-column gel method. The slide method and the test tube method are manually operated with a complicated operation process, and the accuracy of results is greatly influenced by human factors, so that these methods have been gradually eliminated. The microplate method is suitable for large sample size analysis, but it is necessary to provide a large automatic sampler, result judgment is observed with a microscope, and the accuracy of the result judgment is greatly influenced by human factors, so that this method is rarely used in a clinical blood transfusion department. The micro-column gel method is a widely used method at present, but there are also the following limitations: \textcircled{1} this method is insensitive to an ABO weak antigen antibody reaction, and it is found by the inventor that the reason for insensitivity is that a gel card can only be centrifuged once to observe results, but cannot be centrifuged repeatedly to enhance the reaction; \textcircled{2} when a strength of erythrocyte antigen-antibody binding is weak and a shear force produced by the centrifugation of the gel card exceeds an affinity, antibody-dependent erythrocyte clot is separated, leading to false negative results; and \textcircled{3} reagent gel is easy to deform and produce bubbles during transportation, and an air temperature may also affect a size of a gel molecular sieve, thereby affecting the stability and reliability of final test results. The above limitations may lead to inconsistency between forward typing and reverse typing of the micro-column gel method, and at this time, it is necessary to make special treatment on the sample to improve a sensitivity of an immune agglutination reaction, or to make further grouping by a manual method.

Hemolytic disease of the newborn (HDN for short) refers to a blood group incompatibility between a mother and an infant, there is a fetal erythrocyte circulation in the mother, the fetal erythrocyte circulation promotes production of an IgG antibody in the mother, and the IgG antibody can act on fetal erythrocytes through a placenta of the mother, leading to different degrees of hemolysis. In clinic, an incidence of HDN caused by an ABO blood group incompatibility and an RhD blood group incompatibility is high, so that dynamic monitoring of a titer of the IgG antibody in the mother during pregnancy is of great significance for early intervention and treatment of the disease.

At present, an anti-human globulin test determination method is used in all antenatal IgG antibody detections in clinical laboratories, and the determination method is suitable for the test tube method and the micro-column gel method. The test tube method is manually operated, with a complicated operation process, and the accuracy of results is greatly influenced by human factors. The micro-column gel method is widely used in clinical laboratories at present. However, there are some limitations identical to those of the micro-column gel method for ABO blood group reverse typing detection, and these limitations comprise that: \textcircled{1} when a binding strength of erythrocyte antigen and antibody is weak and a shear force produced by the centrifugation of the gel card exceeds an affinity, antibody-dependent erythrocyte clot is separated, leading to false negative results; and \textcircled{2} reagent gel is easy to deform and produce bubbles during transportation, and an air temperature may also affect a size of a gel molecular sieve, thereby affecting the stability and reliability of final test results.

### SUMMARY

Objectives of the present invention: the technical problem to be solved by the present invention is to provide a micro-fluidic chip for blood detection aiming at the defects in the prior art, which is suitable for ABO blood group reverse typing detection and detection of a titer of a blood type IgG antibody of a HDN pregnant woman, and can improve a sensitivity of immune agglutination reaction detection; and meanwhile, the chip has the advantages of small sample usage amount, full automatic detection and trace whole blood separation taking into account hematocrit determination, has a function of “micro-total analysis”, and provides a miniaturized, full automatic and high-throughput supporting analyzer.

In order to solve the above technical problem, a first objective of the present invention discloses a micro-fluidic chip for blood detection. The chip comprises a chip body, the chip body comprises more than one separation and detection unit, and each separation and detection unit comprises a trace blood separation tank for receiving and separating a trace whole blood sample and more than one reaction test unit. When the chip body is rotated, erythrocytes separated from the trace whole blood sample sediment at one end of the trace blood separation tank far away from a center position of the chip body, and plasma separated from the trace whole blood sample is located at the other end of the trace blood separation tank.

Each reaction test unit comprises a first sample loading chamber for receiving the plasma from the trace blood separation tank, a first L-shaped micro-channel, a second sample loading chamber for receiving a corresponding erythrocyte reagent, a second L-shaped micro-channel, a Y-shaped micro-channel and a reaction detection cavity. The Y-shaped micro-channel comprises a first sample injection port, a second sample injection port and an outflow port, and fluids respectively flowing into the first sample injection port and the second sample injection port are mixed through the Y-shaped micro-channel and then discharged from the outflow port. The first L-shaped micro-channel communicates the first sample loading chamber with the first sample injection port of the Y-shaped micro-channel. The second L-shaped micro-channel communicates the second sample loading chamber with the second sample injection port of the Y-shaped micro-channel. The outflow port of the Y-shaped micro-channel is communicated with the reaction detection cavity.

Specifically, the first L-shaped micro-channel and the second L-shaped micro-channel are both composed of a lower-layer micro-channel and a vertical micro-channel which are sequentially communicated, and the lower-layer micro-channel is arranged vertical to the vertical micro-channel. An inlet of the lower-layer micro-channel in the first L-shaped micro-channel is communicated with a bottom portion on one side of the first sample loading chamber far away from the center position of the chip body; and an inlet of the lower-layer micro-channel in the second L-shaped micro-channel is communicated with a bottom portion on one side of the second sample loading chamber far away from the center position of the chip body.

The first sample injection port, the second sample injection port and the outflow port of the Y-shaped micro-channel are all located at an upper portion of the chip body. The first sample injection port of the Y-shaped micro-channel is communicated with an outlet of the vertical micro-channel in the first L-shaped micro-channel, and the second sample injection port of the Y-shaped micro-channel is communicated with an outlet of the vertical micro-channel in the second L-shaped micro-channel.

The Y-shaped micro-channel comprises two upper-layer inlet micro-channels and one upper-layer mixing micro-channel. An inlet of one upper-layer inlet micro-channel is the first sample injection port of the Y-shaped micro-channel, an inlet of the other upper-layer inlet micro-channel is the second sample injection port of the Y-shaped micro-channel, outlets of the two upper-layer inlet micro-channels intersect at an inlet of the upper-layer mixing micro-channel, and an outlet of the upper-layer mixing micro-channel is the outflow port of the Y-shaped micro-channel. Each upper-layer inlet micro-channel is arranged vertical to the corresponding vertical micro-channel.

In some embodiments, the trace blood separation tank is arranged along a radial direction of the chip. The trace blood separation tank comprises a plasma extraction groove and a straight pipe groove communicated with the plasma extraction groove. The straight pipe groove is located at one end of the trace blood separation tank far away from the center position of the chip, and the plasma extraction groove is located at the other end of the trace blood separation tank close to the center position of the chip. Under an action of a centrifugal force, all the erythrocytes obtained after separation of the trace whole blood sample sediment at one end of the straight pipe groove far away from the center position of the chip.

In some embodiments, a surface of the straight pipe groove is provided with a scale mark for interpreting a hematocrit value. A volume of the trace blood separation tank is 50 ul to 100 ul.

In some embodiments, a quantity of the reaction test units is six. The chip further comprises plasma multiple-proportion dilution pretreatment tanks arranged in one-to-one correspondence with the reaction test units.

In some embodiments, the reaction detection cavity comprises a cylindrical chamber and a tapered chamber with a gradually reduced diameter, and a large-diameter end of the tapered chamber is communicated with a bottom end of the cylindrical chamber.

A second objective of the present invention is to provide a detection method of the micro-fluidic chip for blood detection. The detection method comprises the following steps of:

allowing a first micro-fluid of the first sample loading chamber to flow into the first sample injection port of the Y-shaped micro-channel through the first L-shaped micro-channel; and allowing a second micro-fluid of the second sample loading chamber to flow into the second sample injection port of the Y-shaped micro-channel through the second L-shaped micro-channel; and mixing the first micro-fluid and the second micro-fluid in the Y-shaped micro-channel to form a first mixture, and allowing the first mixture to flow into the reaction detection cavity through the outflow port of the Y-shaped micro-channel.

The micro-channel in the micro-fluidic chip is small in scale, a fluid flow in the micro-channel is a laminar flow, and a corresponding Reynolds number is small, so that the mixing between different micro-fluids mainly depends on diffusion. Therefore, in order to enhance the mixing, it is necessary to increase a contact area between solutes, and the contact area can be increased by stretching the fluid or shearing the fluid. Geometric intersection design of pipelines is utilized in the present application, and by arranging the L-shaped micro-channel with a pipeline intersection characteristic and intersecting the vertical micro-channel of the L-shaped micro-channel with the Y-shaped micro-channel in a geometric space, when different micro-fluids respectively flow through the corresponding L-shaped micro-channel and the upper-layer inlet micro-channel of the Y-shaped micro-channel sequentially, the corresponding micro-fluid is divided into many micro-fluid particles first, and then the mixing of micro-fluid particles of different micro-fluids is promoted through the upper-layer mixing micro-channel of the Y-shaped micro-channel.

A third objective of the present invention is to provide a method for carrying out ABO blood group reverse typing detection by using the micro-fluidic chip for blood detection above. A quantity of the reaction test units is three. The erythrocyte reagents received by the second sample adding cavities of the reaction test units are respectively an A-group erythrocyte reagent, a B-group erythrocyte reagent and an O-group erythrocyte reagent. The detection method comprises the following steps:
first step: receiving a to-be-detected trace whole blood sample by the trace blood separation tank;
second step: under the action of the centrifugal force, separating erythrocytes and plasma in the to-be-detected trace whole blood sample, allowing the separated erythrocytes to sediment at one end of the straight pipe groove far away from the center position of the chip, accommodating a first part of separated plasma in the plasma extraction groove, and accommodating a second part of separated plasma in one side of the straight pipe groove close to the center position of the chip;
third step: after centrifugation, sucking the plasma in the plasma extraction groove and transferring the plasma to the first sample loading chamber of each reaction test unit; and receiving the corresponding erythrocyte reagent by the second sample loading chamber of each reaction test unit;
fourth step: under the action of the centrifugal force, allowing the plasma of the first sample loading chamber to flow into the first sample injection port of the Y-shaped micro-channel through the first L-shaped micro-channel; allowing the erythrocyte reagent of the second sample loading chamber to flow into the second sample injection port of the Y-shaped micro-channel through the second L-shaped micro-channel; and mixing the plasma and the erythrocyte reagent in the Y-shaped micro-channel to form a first mixture, and allowing the first mixture to flow into the reaction detection cavity through the outflow port of the Y-shaped micro-channel to fully react in the reaction detection cavity; and
fifth step: allowing the chip to stand, and interpreting to obtain a detection result;
wherein, in a case that a blood group antibody in the to-be-detected plasma is subjected to an immune agglutination reaction with a blood group antigen in the erythrocyte reagent, an erythrocyte clot is formed; under the action of the centrifugal force, the erythrocyte clot sediments in a direction far away from the center position of the chip; and when the chip is stationary, the erythrocyte clot remains adhered to an inner side wall of the reaction detection cavity for a certain period of time; and
in a case that the blood group antibody in the to-be-detected plasma is not subjected to the immune agglutination reaction with the blood group antigen in the erythrocyte reagent, under the action of the centrifugal force, the erythrocytes without the immune agglutination reaction sediment in the direction far away from the center position of the chip; and when the chip is stationary, the erythrocytes without the immune agglutination reaction naturally collapse and sediment due to an action of gravity.

A fourth objective of the present invention is to provide a method for detecting a titer of a blood group IgG antibody of a HDN pregnant woman by using the micro-fluidic chip for blood detection above. A quantity of the reaction test units is six. Each reaction test unit is also correspondingly provided with an independent plasma multiple-proportion dilution pretreatment tank for forming a series of multiple-proportion dilutions. The second sample loading chamber of each reaction test unit receives the same erythrocyte reagent, and the same erythrocyte reagent is an A-group erythrocyte reagent, a B-group erythrocyte reagent or an O-group RhD-positive erythrocyte reagent. The detection method comprises the following steps:
first step: receiving a to-be-detected trace whole blood sample by the trace blood separation tank;
second step: under the action of the centrifugal force, separating erythrocytes and plasma in the to-be-detected trace whole blood sample, allowing the separated erythrocytes to sediment at one end of the straight pipe groove far away from the center position of the chip, accommodating a first part of separated plasma in the plasma extraction groove, and accommodating a second part of separated plasma in one side of the straight pipe groove close to the center position of the chip;
third step: after centrifugation, sucking the plasma in the plasma extraction groove and transferring the plasma to each plasma multiple-proportion dilution pretreatment tank sequentially;
fourth step: pretreating the to-be-detected plasma in each plasma multiple-proportion dilution pretreatment tank with a diluent containing dithiothreitol or 2-mercaptoethanol, and allowing the plasma to stand for reaction for 15 minutes to 30 minutes to destroy an activity of an IgM antibody; and after standing reaction, adding a sample diluent to complete multiple-proportion dilution, so as to obtain plasma subjected to multiple-proportion dilution;
fifth step: sucking the plasma subjected to multiple-proportion dilution in each plasma multiple-proportion dilution pretreatment tank and transferring the plasma subjected to multiple-proportion dilution to the first sample loading chamber of the corresponding reaction test unit; and receiving the erythrocyte reagent by the second sample loading chamber of each reaction test unit;
sixth step: under the action of the centrifugal force, in each reaction test unit, allowing the plasma subjected to multiple-proportion dilution of the first sample loading chamber to flow into the first sample injection port of the Y-shaped micro-channel through the first L-shaped micro-channel; allowing the erythrocyte reagent of the second sample loading chamber to flow into the second sample injection port of the Y-shaped micro-channel through the second L-shaped micro-channel; and mixing the plasma subjected to multiple-proportion dilution and the erythrocyte reagent in the Y-shaped micro-channel to form a first mixture, and allowing the first mixture to flow into the reaction detection cavity through the outflow port of the Y-shaped micro-channel to fully react in the reaction detection cavity; and
seventh step: allowing the chip to stand, and interpreting to obtain a detection result; wherein,
in a case that a blood group antibody in the to-be-detected plasma is subjected to an immune agglutination reaction with a blood group antigen in the erythrocyte reagent, an erythrocyte clot is formed; under the action of the centrifugal force, the erythrocyte clot sediments in a direction far away from the center position of the chip; and when the chip is stationary, the erythrocyte clot remains adhered to an inner side wall of the reaction detection cavity for a certain period of time; and
in a case that the blood group antibody in the to-be-detected plasma is not subjected to the immune agglutination reaction with the blood group antigen in the erythrocyte reagent, under the action of the centrifugal force, the erythrocytes without the immune agglutination reaction sediment in the direction far away from the center position of the chip; and when the chip is stationary, the erythrocytes without the immune agglutination reaction naturally collapse and sediment due to an action of gravity.

Finally, a reciprocal value of a dilution multiple of a plasma sample subjected to highest-multiple-proportion dilution without the immune agglutination reaction is taken as a titer of a blood group IgG antibody against a specific blood group antigen.

Preferably, an anti-human globulin polyclonal antibody lyophilized bead is preset in the reaction detection cavity of each reaction test unit.

### Beneficial Effects:

(1) The present invention provides the micro-fluidic chip for blood detection, which is suitable for the ABO blood group reverse typing detection and the detection of the titer of the blood group IgG antibody of the HDN pregnant woman. The micro-fluidic chip for blood detection of the present application has a function of “micro-total analysis”, different from a test tube method and a micro-column gel method which need to be completed by multiple steps and multiple vessels, the present application completes all processes of mixing, entering the reaction cavity, centrifugally accelerating the reaction, stopping centrifugation and standing, presenting reaction results, etc. in one separation and detection unit of one micro-fluidic chip for blood detection, which simplifies an operation process, and provides realization conditions for a miniaturized, full automatic and high-throughput supporting analyzer.
(2) The present invention can complete the detection through the trace whole blood sample. A volume of the trace blood separation tank is set to be 50 ul to 100 ul, and a sample injection volume is 40 ul to 80 ul, which can well solve the applicability problem of people with blood collection difficulty, especially newborns, and is conducive to saving reagent costs at the same time.
(3) The micro-fluidic chip for blood detection of the present invention belongs to a centrifugal micro-fluidic chip, which is provided with the L-shaped micro-channel and the Y-shaped micro-channel. Under the action of the centrifugal force, the first micro-fluid of the first sample loading chamber flows into the first sample injection port of the Y-shaped micro-channel through the first L-shaped micro-channel. The second micro-fluid of the second sample loading chamber flows into the second sample injection port of the Y-shaped micro-channel through the second L-shaped micro-channel. The first micro-fluid and the second micro-fluid are mixed in the Y-shaped micro-channel to form the first mixture, and the first mixture flows into the reaction detection cavity through the outflow port of the Y-shaped micro-channel. Geometric intersection design of pipelines is utilized in the present application, and by arranging the L-shaped micro-channel with a pipeline intersection characteristic and intersecting the vertical micro-channel of the L-shaped micro-channel with the upper-layer inlet micro-channel of the Y-shaped micro-channel in a geometric space, when different micro-fluids respectively flow through the corresponding L-shaped micro-channel and the upper-layer inlet micro-channel of the Y-shaped micro-channel sequentially, the corresponding micro-fluid is divided into many micro-fluid particles first, and then the mixing of micro-fluid particles of different micro-fluids is promoted through the upper-layer mixing micro-channel of the Y-shaped micro-channel. In the present application, the above mixing process is used for mixing an erythrocyte suspension with other fluids such as plasma, and the erythrocyte suspension and the plasma are fully mixed to react by a principle of fluid mechanics inside the micro-fluidic chip, so as to meet conditions of the immune agglutination reaction. Compared with other mixing methods, such as magnetic stirring, the mixing method in the present application has the advantages of no residual contamination, no need to add additional materials, no need for structures such as a magnetic stirring module, being beneficial for simplifying the structure of the supporting analyzer, etc.
(4) The micro-fluidic chip for blood detection of the present invention allows the weak antigen-antibody immune agglutination reaction to be enhanced by repeated centrifugation, thereby improving a sensitivity of weak antigen antibody reaction detection, that is, improving a sensitivity of ABO blood group system reverse typing detection. In a traditional micro-column gel method, under the action of the centrifugal force, erythrocyte clot inside a gel card may be subjected to a shear stress, and when the shear stress is greater than an affinity of erythrocyte antigen-antibody binding, the antibody-dependent erythrocyte clot will be separated, leading to a false negative test result. In order to avoid the false negative test result, the micro-column gel method limits that the test result is interpreted only by centrifuging the gel card once. Although this method ensures a specificity to the weak antigen-antibody reaction detection, the sensitivity of the weak antigen-antibody reaction detection is reduced. Different from the traditional micro-column gel method, if the erythrocyte antigen-antibody binding occurs in the micro-fluidic chip for blood detection of the present application, the erythrocyte clot is relatively stable without the influence of the shear stress during centrifugation, and there is no risk of the false negative result in the micro-column gel method, so that the micro-fluidic chip for blood detection of the present application allows the weak antigen-antibody immune agglutination reaction to be enhanced by repeated centrifugation. If the immune agglutination reaction occurs and the reaction is sufficient, under the action of the centrifugal force, the erythrocyte clot sediments and is adhered to an inner side wall of the reaction cavity. The erythrocyte clot will not naturally collapse and sediment after standing for a certain period of time, and will remain vertically adhered to the wall of the reaction cavity. There is no visible sedimented erythrocyte adhesion layer at a bottom portion of the reaction cavity, which indicates that the plasma contains an erythrocyte blood group antibody, that is, the result is positive. If there is no immune agglutination reaction, the centrifugal force is removed, and after standing for a period of time, the erythrocytes without the immune agglutination reaction naturally collapse and sediment, and gather in the center of the bottom portion of the reaction cavity along an inverted conical slope at the bottom portion of the reaction cavity to form the erythrocyte adhesion layer, which indicates that there is no erythrocyte blood group antibody in the plasma, that is, the result is negative.
(5) When the titer of the blood group IgG antibody of the HDN pregnant woman is detected by the chip of the present invention, because there is no shear stress of the micro-column gel method in the micro-channel and the reaction cavity of the chip during centrifugation, the method for detecting the titer of the blood group IgG antibody of the HDN pregnant woman by using the chip of the present invention avoids the risk of the false negative result of the micro-column gel method.
(6) When the titer of the blood group IgG antibody of the HDN pregnant woman is detected by the chip of the present invention, the anti-human globulin polyclonal antibody lyophilized bead may be preset in the reaction detection cavity. A stability of a lyophilized bead reagent is better than that of a liquid reagent at room temperature, so that a transportation process and a temperature have little influence on a performance stability of the chip.
(7) In the present invention, whether the immune agglutination reaction occurs may be interpreted by microscopic image photography in combination with artificial intelligence, which reduces influences of human factors on the interpretation and simplifies operation steps; and meanwhile, images may be permanently stored to facilitate rechecking, which improves the accuracy of test results.
(8) In the present application, the trace blood separation tank comprises the plasma extraction groove and the straight pipe groove communicated with the plasma extraction groove in structure. By arranging the straight pipe groove, the trace blood separation tank not only has the function of trace whole blood separation, but also has the function of determining the hematocrit. The surface of the straight pipe groove is provided with the scale mark for interpreting the hematocrit, which facilitates manually reading a value of the hematocrit as a reference for blood transfusion.

### BRIEF DESCRIPTION OF THE DRAWINGS

The present invention is further described in detail hereinafter with reference to the drawings and specific embodiments, and the advantages of the above and/or other aspects of the present invention will become clearer.

FIG. 1 is a schematic diagram of a stereoscopic structure of a chip body of a micro-fluidic chip for blood detection used for ABO blood group reverse typing detection in First Embodiment of the present application;

FIG. 2 is a top view of an upper chip layer of the micro-fluidic chip for blood detection used for ABO blood group reverse typing detection in First Embodiment of the present application;

FIG. 3 is a top view of the chip body as shown in FIG. 1;

FIG. 4 is a partial enlarged drawing of one separation and detection unit in the chip body as shown in FIG. 3;

FIG. 5 is a stereoscopic diagram of a cross section in a direction A as shown in FIG. 3;

FIG. 6 is a schematic diagram of a stereoscopic structure of a chip body of a micro-fluidic chip for blood detection used for detecting a titer of a blood group IgG antibody of a HDN pregnant woman in Second Embodiment of the present application;

FIG. 7 is a top view of an upper chip layer of the micro-fluidic chip for blood detection used for detecting the titer of the blood group IgG antibody of the HDN pregnant woman in Second Embodiment of the present application;

FIG. 8 is a top view of the chip body as shown in FIG. 6; and

FIG. 9 is a partial enlarged drawing of one separation and detection unit in the chip body as shown in FIG. 8.

Reference numerals are as follows:
1 refers to chip body; 101 refers to first separation and detection unit; 102 refers to second separation and detection unit; 110 refers to trace blood separation tank; 111 refers to plasma extraction groove; 112 refers to straight pipe groove; 120 refers to first sample loading chamber; 130 refers to first L-shaped micro-channel; 140 refers to second sample loading chamber; 150 refers to second L-shaped micro-channel; 160 refers to Y-shaped micro-channel; 161 refers to first sample injection port; 162 refers to second sample injection port; 163 refers to outflow port; 170 refers to reaction detection cavity; 180a, 180b, 180c, 180d, 180e and 180f refer to multiple-proportion dilution pretreatment tanks; 2 refers to upper chip layer; 201 refers to trace blood separation tank injection hole; 202 refers to first sample loading chamber injection hole; 203 refers to second sample loading chamber injection hole; and 204a, 204b, 204c, 204d, 204e and 204f refer to plasma multiple-proportion dilution pretreatment tank injection holes.

### DETAILED DESCRIPTION

Technical solutions of the present application are described in detail hereinafter with reference to the drawings.

### Embodiment 1

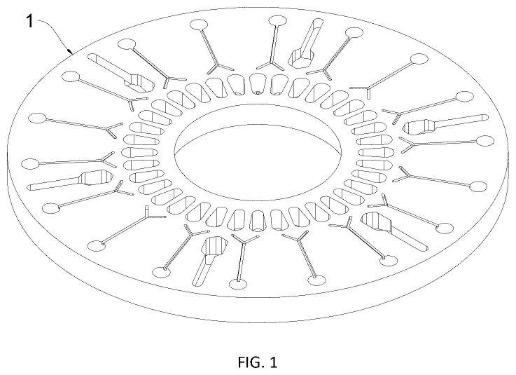

This embodiment provides a micro-fluidic chip for blood detection, and the micro-fluidic chip for blood detection is used for ABO blood group reverse typing detection. The chip comprises a chip body 1 and an upper chip layer 2. FIG. 1 provides a schematic diagram of a stereoscopic structure of the chip body 1 in this embodiment.

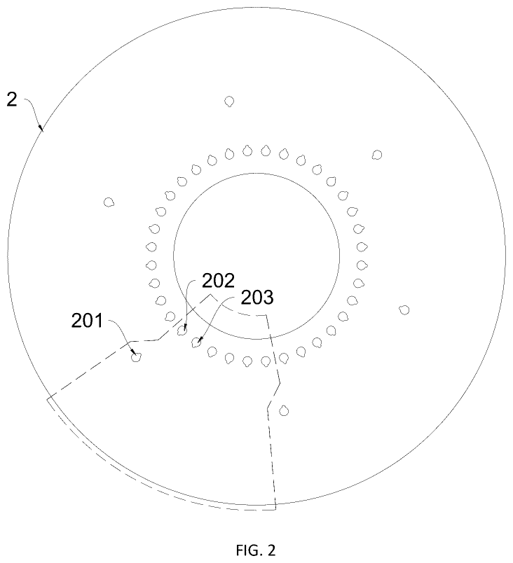

FIG. 2 provides a top view of the upper chip layer 2 in this embodiment. The upper chip layer 2 may be a transparent film covering a top surface of the chip body 1.

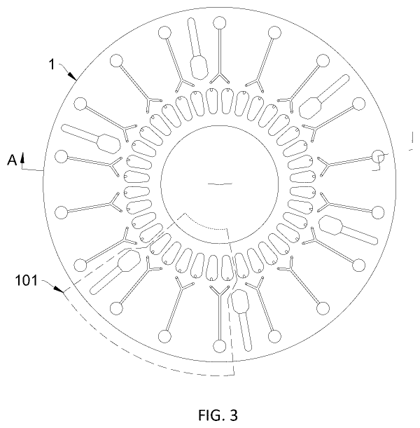

FIG. 3 provides a top view of the chip body 1 as shown in FIG. 1. As shown in FIG. 3, the chip body 1 comprises six separation and detection units. The six separation and detection units are evenly distributed along a circumferential direction of a central rotating axis of the chip body 1. The separation and detection unit in this embodiment is equivalent to a first separation and detection unit 101 in FIG. 3.

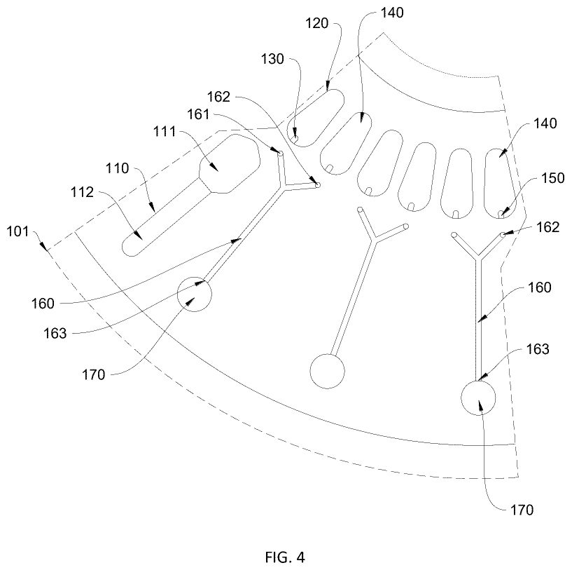

FIG. 4 provides a partial enlarged drawing of one separation and detection unit in the chip body as shown in FIG. 3, wherein a dotted line area represents one separate detection unit. As shown in FIG. 4, each first separation and detection unit 101 comprises a trace blood separation tank 110 for receiving and separating a trace whole blood sample and three reaction test units. A volume of the trace blood separation tank 110 is fixed, and may be set to be 50 ul to 100 ul, and a sample injection volume is 40 ul to 80 ul, which can well solve the applicability problem of people with blood collection difficulty, especially newborns. When the chip body 1 is rotated at a velocity of 2000 rpm to 5000 rpm, erythrocytes separated from the trace whole blood sample sediment at one end of the trace blood separation tank 110 far away from a center position of the chip body 1, and plasma separated from the trace whole blood sample is located at the other end of the trace blood separation tank 110.

As shown in FIG. 4, each reaction test unit comprises a first sample loading chamber 120 for receiving the plasma from the trace blood separation tank 110, a first L-shaped micro-channel 130, a second sample loading chamber 140 for receiving a corresponding erythrocyte reagent, a second L-shaped micro-channel 150, a Y-shaped micro-channel 160 and a reaction detection cavity 170. In this embodiment, the erythrocyte reagents received by the second sample adding cavities 140 of the three reaction test units are respectively an A-group erythrocyte reagent, a B-group erythrocyte reagent and an O-group erythrocyte reagent. In the same reaction test unit, the first sample loading chamber 120 and the second sample loading chamber 140 are adjacent to each other and located on the same concentric circle of the central rotating axis of the chip body 1. The first sample loading chamber 120 and the second sample loading chamber 140 are closer to the center position of the chip than the reaction detection cavity 170.

As shown in FIG. 4, the Y-shaped micro-channel 160 comprises a first sample injection port 161, a second sample injection port 162 and an outflow port 163. Fluids respectively flowing into the first sample injection port 161 and the second sample injection port 162 are mixed through the Y-shaped micro-channel 160 and then discharged from the outflow port 163. The first L-shaped micro-channel 130 communicates the first sample loading chamber 120 with the first sample injection port 161 of the Y-shaped micro-channel 160. The second L-shaped micro-channel 150 communicates the second sample loading chamber 140 with the second sample injection port 162 of the Y-shaped micro-channel 160. The outflow port 163 of the Y-shaped micro-channel 160 is communicated with the reaction detection cavity 170.

During detection, under an action of a centrifugal force, a first micro-fluid of the first sample loading chamber 120 flows into the first sample injection port 161 of the Y-shaped micro-channel 160 through the first L-shaped micro-channel 130. A second micro-fluid of the second sample loading chamber 140 flows into the second sample injection port 162 of the Y-shaped micro-channel 160 through the second L-shaped micro-channel 150. The first micro-fluid and the second micro-fluid are mixed in the Y-shaped micro-channel 160 to form a first mixture, and the first mixture flows into the reaction detection cavity 170 through the outflow port 163 of the Y-shaped micro-channel 160. The first micro-fluid and the second micro-fluid may both be a liquid, or at least one of the first micro-fluid and the second micro-fluid is a multiphase mixture. In a specific embodiment, the first micro-fluid is plasma and the second first micro-fluid is an erythrocyte suspension. Under an action of a centrifugal force of 300 rpm to 2000 rpm, the plasma and the erythrocyte suspension respectively flow into the Y-shaped micro-channel 160 through corresponding L-shaped micro-channels. By a mixing function of the Y-shaped micro-channel 160, the plasma and the erythrocyte suspension are mixed to fully react. The multiphase mixture formed after the mixing reaction enters and fills the reaction detection cavity 170.

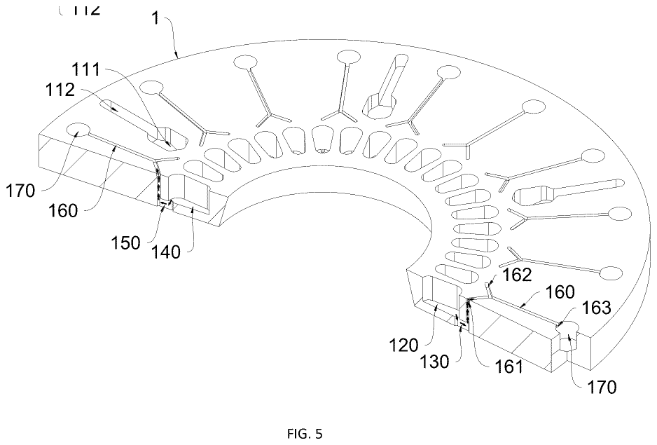

FIG. 5 provides a stereoscopic diagram of a cross section in a direction A as shown in FIG. 3, wherein an arrow in the figure indicates a flow direction of the fluid under the action of the centrifugal force. As shown in FIG. 5, the first L-shaped micro-channel 130 and the second L-shaped micro-channel 150 are both composed of a lower-layer micro-channel and a vertical micro-channel which are sequentially communicated, and the lower-layer micro-channel is arranged vertical to the vertical micro-channel. The lower-layer micro-channel is vertically connected with the vertical micro-channel to achieve a pipeline intersection characteristic of the L-shaped micro-channel. The lower-layer micro-channel is provided with a sealing film to prevent the sample from leaking out. An inlet of the lower-layer micro-channel in the first L-shaped micro-channel 130 is communicated with a bottom portion on one side of the first sample loading chamber 120 far away from the center position of the chip body 1. An inlet of the lower-layer micro-channel in the second L-shaped micro-channel 150 is communicated with a bottom portion on one side of the second sample loading chamber 140 far away from the center position of the chip body 1.

The first sample injection port 161, the second sample injection port 162 and the outflow port 163 of the Y-shaped micro-channel 160 are all located at an upper portion of the chip body 1. The first sample injection port 161 of the Y-shaped micro-channel 160 is communicated with an outlet of the vertical micro-channel in the first L-shaped micro-channel 130, and the second sample injection port 162 of the Y-shaped micro-channel 160 is communicated with an outlet of the vertical micro-channel in the second L-shaped micro-channel 150.

Specifically, as shown in FIG. 5, the Y-shaped micro-channel 160 comprises two upper-layer inlet micro-channels and one upper-layer mixing micro-channel. An inlet of one upper-layer inlet micro-channel is the first sample injection port 161 of the Y-shaped micro-channel 160, and an inlet of the other upper-layer inlet micro-channel is the second sample injection port 162 of the Y-shaped micro-channel 160. Outlets of the two upper-layer inlet micro-channels intersect at an inlet of the upper-layer mixing micro-channel. An outlet of the upper-layer mixing micro-channel is the outflow port 163 of the Y-shaped micro-channel 160. The Y-shaped micro-channel 160 may be located in a plane perpendicular to the vertical micro-channel, which facilitates arranging each upper-layer inlet micro-channel vertical to the corresponding vertical micro-channel.

Geometric intersection design of pipelines is utilized in the present application, and by arranging the L-shaped micro-channel with a pipeline intersection characteristic and intersecting the vertical micro-channel of the L-shaped micro-channel with the Y-shaped micro-channel in a geometric space, when different micro-fluids respectively flow through the corresponding L-shaped micro-channel and the upper-layer inlet micro-channel of the Y-shaped micro-channel sequentially, the corresponding micro-fluid is divided into many micro-fluid particles first, and then the mixing of micro-fluid particles of different micro-fluids is promoted through the upper-layer mixing micro-channel of the Y-shaped micro-channel.

As shown in FIG. 5, the trace blood separation tank 110 is arranged along a radial direction of the chip. In order to have a function of hematocrit detection at the same time, as shown in FIG. 5, the trace blood separation tank 110 comprises a plasma extraction groove 111 and a straight pipe groove 112 communicated with the plasma extraction groove 111. The straight pipe groove 112 is located at one end of the trace blood separation tank 110 far away from the center position of the chip, and the plasma extraction groove 111 is located at the other end of the trace blood separation tank 110 close to the center position of the chip. Specifically, the plasma extraction groove 111 in the trace blood separation tank 110 is used for receiving a to-be-detected trace whole blood sample. Under the action of the centrifugal force, erythrocytes separated from the trace whole blood sample all sediment at one end of the straight pipe groove 112 far away from the center position of the chip, a first part of plasma is accommodated in the plasma extraction groove 111, and a second part of plasma is accommodated in one end of the straight pipe groove 112 close to the center position of the chip. In some examples, when the chip body 1 is rotated at a velocity of 2000 rpm to 5000 rpm, trace whole blood in the trace blood separation tank 110 is separated.

In order to facilitate manual interpretation, a surface of the straight pipe groove 112 may be provided with a scale mark for interpreting a hematocrit value. The scale mark is not shown in the figure.

In order to improve the interpretation sensitivity and accuracy to the reaction detection cavity, as shown in FIG. 5, the reaction detection cavity 170 comprises a cylindrical chamber and a tapered chamber with a gradually reduced diameter. A large-diameter end of the tapered chamber is communicated with a bottom end of the cylindrical chamber.

As shown in FIG. 2, one end of a top portion of the trace blood separation tank 110 close to the center position of the chip body 1 is provided with a trace blood separation tank injection hole 201 for adding the trace whole blood sample. One end of a top portion of the first sample loading chamber 120 close to the center position of the chip body 1 is provided with a first sample loading chamber injection hole 202 for adding the plasma from the trace blood separation tank 110. One end of a top portion of the second sample loading chamber 140 close to the center position of the chip body 1 is provided with a second sample loading chamber injection hole 203 for adding the corresponding erythrocyte reagent. The trace blood separation tank injection hole 201, the first sample loading chamber injection hole 202 and the second sample loading chamber injection hole 203 are all located in the upper chip layer 2.

Preferably, as shown in FIG. 2, the trace blood separation tank injection hole 201, the first sample loading chamber injection hole 202 and the second sample loading chamber injection hole 203 are all provided with a notch for ventilation, which achieves an effect that when the sample is added, the notch is beneficial for exhausting air, so as to avoid the sample from overflowing the hole. The notch may be U-shaped or V-shaped.

A method for carrying out ABO blood group reverse typing detection by using the micro-fluidic chip for blood detection in this embodiment comprises the following steps.

In first step, a to-be-detected trace whole blood sample is received by the trace blood separation tank 110.

In second step, under the action of the centrifugal force, erythrocytes and plasma in the to-be-detected trace whole blood sample are separated, the separated erythrocytes sediment at one end of the straight pipe groove 112 far away from the center position of the chip, a first part of separated plasma is accommodated in the plasma extraction groove 111, and a second part of separated plasma is accommodated in one side of the straight pipe groove 112 close to the center position of the chip.

In third step, after centrifugation, the plasma in the plasma extraction groove 111 is sucked and transferred to the first sample loading chamber 120 of each reaction test unit. The corresponding erythrocyte reagent is received by the second sample loading chamber 140 of each reaction test unit.

In fourth step, under the action of the centrifugal force, the plasma of the first sample loading chamber 120 flows into the first sample injection port 161 of the Y-shaped micro-channel 160 through the first L-shaped micro-channel 130; and the erythrocyte reagent of the second sample loading chamber 140 flows into the second sample injection port 162 of the Y-shaped micro-channel 160 through the second L-shaped micro-channel 150. Under the action of the centrifugal force, the plasma and the erythrocyte reagent are mixed in the Y-shaped micro-channel 160 to form a first mixture, and the first mixture flows into the reaction detection cavity 170 through the outflow port 163 of the Y-shaped micro-channel 160 to fully react for 1 minute to 5 minutes in the corresponding reaction detection cavity 170. The action of the centrifugal force can shorten a distance between erythrocytes, promote an immune agglutination reaction between an antibody and an erythrocyte antigen, and enhance an intensity of the immune agglutination reaction.

In fifth step, the micro-fluidic chip for blood detection is controlled to stop rotating, and the chip stands still and is interpreted to obtain a detection result.

In this embodiment, an interpretation principle of whether the immune agglutination reaction occurs in each reaction detection cavity 170 is as follows: in a case that a blood group antibody in the to-be-detected plasma is subjected to the immune agglutination reaction with a blood group antigen in the erythrocyte reagent, an erythrocyte clot is formed. Under the action of the centrifugal force, the erythrocyte clot may be vertically adhered to an inner side wall of the reaction detection cavity 170 due to centrifugal sedimentation. After the micro-fluidic chip for blood detection is controlled to stop rotating, the erythrocyte clot will not naturally collapse for a certain period of time under a standing state of the chip body, that is, the erythrocyte clot remains adhered to the inner side wall of the reaction detection cavity 170 for a certain period of time. There are no unagglutinated erythrocytes at a bottom portion of the reaction detection cavity 170, which indicates that the result is positive.

In a case that the blood group antibody in the to-be-detected plasma is not subjected to the immune agglutination reaction with the blood group antigen in the erythrocyte reagent, under the action of the centrifugal force, the erythrocytes without the immune agglutination reaction may also be vertically adhered to the inner side wall of the reaction detection cavity 170 due to centrifugal sedimentation. However, different from the erythrocyte clot, the erythrocytes without the immune agglutination reaction may naturally collapse and sediment due to an action of gravity after standing for a period of time, that is, a large number of unagglutinated erythrocytes are formed at the bottom portion of the reaction detection cavity 170, which indicates that the result is negative.

For an ABO blood group and an Rh weak D antigen, in a standing process after first centrifugation, a sedimentation amount or a sedimentation velocity of the erythrocyte at the bottom portion of the reaction detection cavity 170 is obviously lower than that of a control reaction cavity. The control reaction cavity refers to a reaction detection cavity in the reaction test unit added with the O-group erythrocyte reagent. At this time, the chip of the present application may be repeatedly centrifuged for 2 to 3 times to directly enhance the immune agglutination reaction. Compared with a gel method, the chip of the present application can improve the sensitivity of the immune agglutination detection by repeated centrifugation, so as to avoid forward and reverse typing inconsistency caused by a weak antibody in the traditional micro-column gel method.

In this embodiment, the detection results may be obtained by interpretation through naked eyes or by microphotography combined with artificial intelligence analysis. Compared with the interpretation through naked eyes, the microphotography combined with artificial intelligence analysis reduces an interference of human factors, and allows recheck because images can be permanently stored, thereby being conductive to improving the accuracy of the detection results.

In this embodiment, because the bottom portion of the reaction detection cavity 170 is provided with the tapered chamber, the unagglutinated erythrocytes gather at a conical tip to form a sedimented erythrocyte adhesion layer. The sedimented erythrocyte adhesion layer provides a clearer, more readable, more sensitive and more accurate interpretation method.

Interpretation of reverse-typing blood grouping results is as shown in Table 1.

TABLE 1
Interpretation of reverse-typing blood grouping results

| A-group | B-group | O-group | |
|---|---|---|---|
| erythrocyte | erythrocyte | erythrocyte | Reverse-typing blood |
| reagent | reagent | reagent | grouping result |
| Negative | Positive | Negative | A-group (anti-A antibody) |
| Positive | Negative | Negative | B-group (anti-B antibody) |
| Negative | Negative | Negative | AB-group (without anti-A |
| | | | antibody and anti-B antibody) |
| Positive | Positive | Negative | O-group (anti-A antibody and |
| | | | anti-B antibody) |

### Embodiment 2

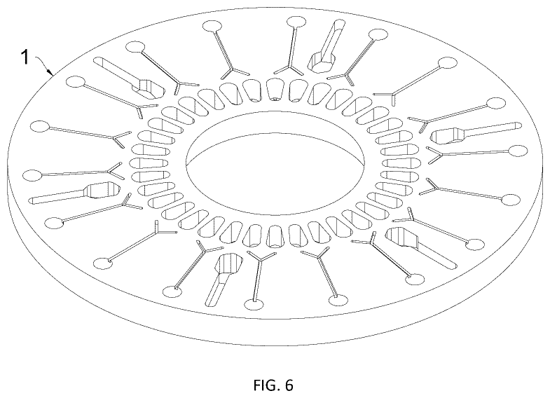

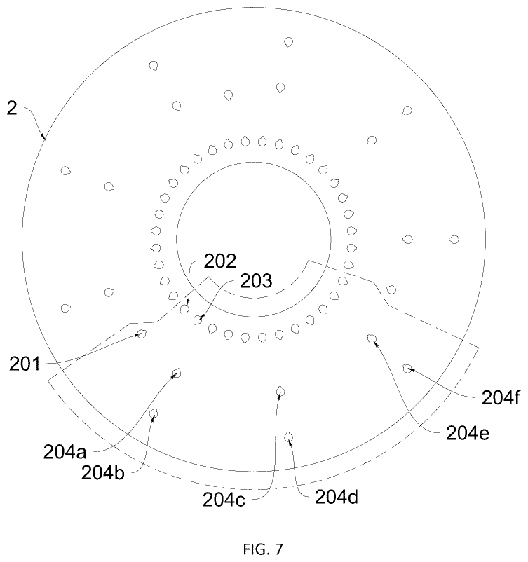

This embodiment provides a micro-fluidic chip for blood detection, and the micro-fluidic chip for blood detection is used for detecting a titer of a blood group IgG antibody of a HDN pregnant woman. The chip comprises a chip body 1 and an upper chip layer 2 covering a top surface of the chip body 1. FIG. 6 provides a schematic diagram of a stereoscopic structure of the chip body 1 of the micro-fluidic chip for blood detection in this embodiment. FIG. 7 provides a top view of the upper chip layer 2 in this embodiment.

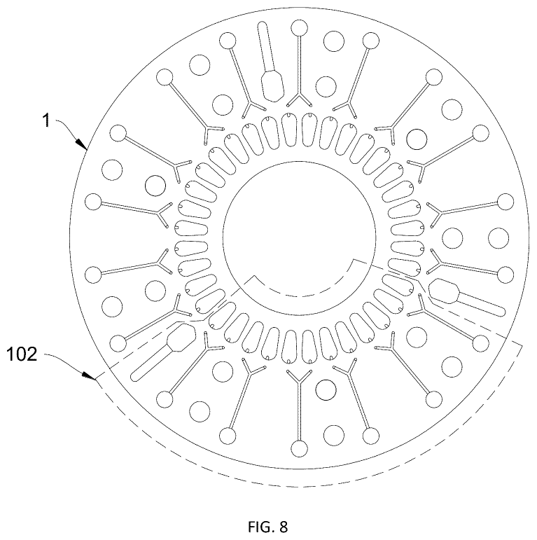

FIG. 8 provides a top view of the chip body 1 as shown in FIG. 6. As shown in FIG. 8, the chip body 1 in this embodiment comprises three separation and detection units, and the three separation and detection units are evenly distributed along a circumferential direction of a central rotating axis of the chip body 1. Each separation and detection unit in this embodiment is equivalent to a second separation and detection unit 102 in FIG. 8.

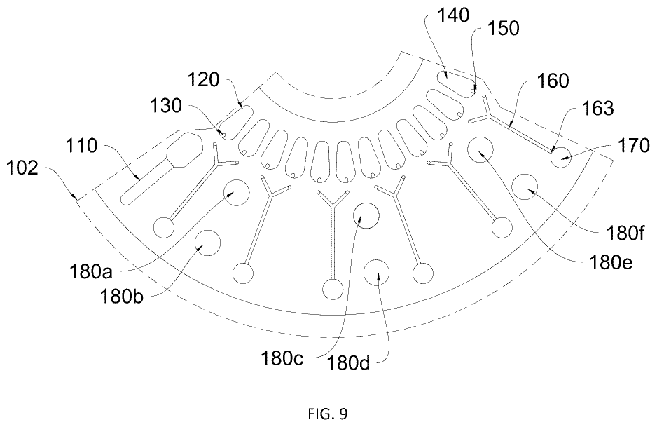

FIG. 9 provides a partial enlarged drawing of the second separation and detection unit 102 in the chip body 1 as shown in FIG. 8, wherein a dotted line area represents one separate detection unit. As shown in FIG. 9, different from Embodiment 1, in each second separation and detection unit 102 in this embodiment, a quantity of reaction test units is six. In addition, the chip body 1 in this embodiment further comprises plasma multiple-proportion dilution pretreatment tanks 180a, 180b, 180c, 180d, 180e and 180f which are arranged in one-to-one correspondence with the reaction test units for preparing a series of plasma with different dilution multiples, so that when a user uses the chip in this embodiment, it is unnecessary to prepare a plasma multiple-proportion diluent preparation test tube, thereby being conductive to saving consumables at the same time. Generally, plasma with dilution multiples of 2, 4, 8, 16, 32 and 64 is used for determining a titer of an anti-Rh blood group IgG antibody, and plasma with dilution multiples of 64, 128, 256, 512, 1024 and 2048 is used for determining titers of anti-A and anti-B blood group IgG antibodies.

In this embodiment, the second sample loading chamber 140 of each reaction test unit receives the same erythrocyte reagent, and the same erythrocyte reagent is an A-group erythrocyte reagent, a B-group erythrocyte reagent or an O-group RhD-positive erythrocyte reagent. The A-group erythrocyte reagent and the B-group erythrocyte reagent are suitable for detecting a titer of a blood group IgG antibody of a HDN pregnant woman inconsistent with the ABO blood group. The O-group RhD-positive erythrocyte reagent is suitable for detecting a titer of a blood group IgG antibody of a HDN pregnant woman inconsistent with the Rh blood group.

Specifically, a possible blood group of a fetus is predicted according to blood groups of parents, and then a corresponding known blood group erythrocyte reagent is selected to determine a corresponding antibody in maternal blood. For example, if the pregnant woman has A-group RhD-negative blood and the father has B-group RhD-positive blood, the fetus may have A-group RhD-positive blood, A-group RhD-negative blood, B-group RhD-positive blood, B-group RhD-negative blood, AB-group RhD-positive blood, AB-group RhD-negative blood, O-group RhD-positive blood or O-group RhD-negative blood, and in this case, it is necessary to select the B-group erythrocyte reagent and the O-group RhD-positive erythrocyte reagent to detect anti-B and anti-RhD antibodies of the pregnant woman respectively. If the father has B-group RhD-negative blood, the fetus may have A-group RhD-negative blood, B-group RhD-negative blood, AB-group RhD-negative blood or O-group RhD-negative blood, so that it is only necessary to select the B-group erythrocyte reagent to detect the anti-B antibody of the pregnant woman, that is, only one separation and detection unit is needed.

As shown in FIG. 7, in this embodiment, in addition to the trace blood separation tank injection hole 201, the first sample loading chamber injection hole 202 and the second sample loading chamber injection hole 203, the upper chip layer 2 is further provided with six plasma multiple-proportion dilution pretreatment tank injection holes 204a, 204b, 204c, 204d, 204e and 204f. The plasma multiple-proportion dilution pretreatment tank injection holes 204a to 204f are sequentially arranged in top portions of the plasma multiple-proportion dilution pretreatment tanks 180a to 180f. For example, the plasma multiple-proportion dilution pretreatment tank injection hole 204a is located in the top portion of the plasma multiple-proportion dilution pretreatment tank 180a, the plasma multiple-proportion dilution pretreatment tank injection hole 204b is located in the top portion of the plasma multiple-proportion dilution pretreatment tank 180b, and so on.

A method for detecting a titer of a blood group IgG antibody of a HDN pregnant woman by using the micro-fluidic chip for blood detection in this embodiment comprises the following steps.

In first step, a to-be-detected trace whole blood sample is received by the trace blood separation tank 110.

In second step, under the action of the centrifugal force, erythrocytes and plasma in the to-be-detected trace whole blood sample are separated, the separated erythrocytes sediment at one end of the straight pipe groove 112 far away from the center position of the chip, a first part of separated plasma is accommodated in the plasma extraction groove 111, and a second part of separated plasma is accommodated in one side of the straight pipe groove 112 close to the center position of the chip.

In third step, after centrifugation, the plasma in the plasma extraction groove 111 is sucked and transferred to each plasma multiple-proportion dilution pretreatment tank sequentially.

In fourth step, the plasma in the plasma multiple-proportion dilution pretreatment tank is pretreated with a diluent containing dithiothreitol (DTT) or 2-mercaptoethanol (2-ME), and the plasma stands for reaction for 15 minutes to 30 minutes to destroy an activity of an IgM antibody. After standing reaction, a sample diluent is added to complete multiple-proportion dilution, so as to obtain plasma subjected to multiple-proportion dilution. The sample diluent may be a PBS (phosphate buffer solution) or normal saline.

In fifth step, the plasma subjected to multiple-proportion dilution in each plasma multiple-proportion dilution pretreatment tank is sucked and transferred to the first sample loading chamber 120 of the corresponding reaction test unit; and the erythrocyte reagent is received by the second sample loading chamber 140 of each reaction test unit.

In sixth step, under the action of the centrifugal force, in each reaction test unit, the plasma subjected to multiple-proportion dilution of the first sample loading chamber 120 flows into the first sample injection port 161 of the Y-shaped micro-channel 160 through the first L-shaped micro-channel 130; the erythrocyte reagent of the second sample loading chamber 140 flows into the second sample injection port 162 of the Y-shaped micro-channel 160 through the second L-shaped micro-channel 150; and under the action of the centrifugal force, the plasma subjected to multiple-proportion dilution and the erythrocyte reagent are mixed in the Y-shaped micro-channel 160 to form a first mixture, and the first mixture flows into the reaction detection cavity 170 through the outflow port 163 of the Y-shaped micro-channel 160 to fully react for 1 minute to 5 minutes in the reaction detection cavity 170.

In seventh step, the micro-fluidic chip for blood detection is controlled to stop rotating, and the chip stands still and is interpreted to obtain a detection result.

In this embodiment, an interpretation principle of whether an immune agglutination reaction occurs in each reaction detection cavity 170 is the same as that in First Embodiment of the present application. In a case that a blood group antibody in the plasma subjected to multiple-proportion dilution is subjected to the immune agglutination reaction with a blood group antigen in the erythrocyte reagent, an erythrocyte clot is formed, and under the action of the centrifugal force, the erythrocyte clot may be vertically adhered to the inner side wall of the reaction detection cavity 170 due to centrifugal sedimentation. The erythrocyte clot will not naturally collapse during standing for a period of time, that is, the erythrocyte clot remains adhered to the inner side wall of the reaction detection cavity 170. There are no unagglutinated erythrocytes at a bottom portion of the reaction detection cavity 170, which indicates that the result is positive.

In a case that the blood group antibody in the plasma subjected to multiple-proportion dilution is not subjected to the immune agglutination reaction with the blood group antigen in the erythrocyte reagent, under the action of the centrifugal force, the erythrocytes without the immune agglutination reaction may also be vertically adhered to the inner side wall of the reaction detection cavity 170 due to centrifugal sedimentation. However, different from the erythrocyte clot, the erythrocytes without the immune agglutination reaction may naturally collapse and sediment due to an action of gravity after standing for a period of time, that is, unagglutinated erythrocytes are formed at the bottom portion of the reaction detection cavity 170, which indicates that the result is negative. A reciprocal value of a dilution multiple of a plasma sample subjected to highest-multiple-proportion dilution without the immune agglutination reaction is taken as a titer of a blood group IgG antibody against a specific blood group antigen.

In this embodiment, an anti-human globulin polyclonal antibody lyophilized bead may be preset in the reaction detection cavity 170 of each reaction test unit. A stability of a lyophilized bead reagent is better than that of a liquid reagent at room temperature. An anti-human globulin antibody serving as a second antibody plays a role of bridge to link the specific antibody bound with the erythrocyte antigen, so as to agglutinate the erythrocytes.

In this embodiment, because the bottom portion of the reaction detection cavity 170 is provided with the tapered chamber, the unagglutinated erythrocytes gather at a conical tip to form a sedimented erythrocyte adhesion layer. The sedimented erythrocyte adhesion layer provides a clearer, more readable, more sensitive and more accurate interpretation method.

The present invention provides an idea and a method for a micro-fluidic chip for blood detection and a detection method thereof, with many methods and ways to realize the technical solution specifically. Those described above are merely the preferred embodiments of the present invention, and it should be pointed out that those of ordinary skills in the art may further make improvements and decorations without departing from the principle of the present invention, and these improvements and decorations should also be regarded as the scope of protection of the present invention. All the unspecified components in the embodiments can be realized by the prior art.

1. A micro-fluidic chip for blood detection, wherein the chip comprises a chip body (1), the chip body (1) comprises more than one separation and detection unit, and each separation and detection unit comprises a trace blood separation tank (110) for receiving and separating a trace whole blood sample and more than one reaction test unit; when the chip body (1) is rotated, erythrocytes separated from the trace whole blood sample sediment at one end of the trace blood separation tank (110) far away from a center position of the chip body (1), and plasma separated from the trace whole blood sample is located at the other end of the trace blood separation tank (110); each reaction test unit comprises a first sample loading chamber (120) for receiving the plasma from the trace blood separation tank (110), a first L-shaped micro-channel (130), a second sample loading chamber (140) for receiving a corresponding erythrocyte reagent, a second L-shaped micro-channel (150), a Y-shaped micro-channel (160) and a reaction detection cavity (170); the Y-shaped micro-channel (160) comprises a first sample injection port (161), a second sample injection port (162) and an outflow port (163), and fluids respectively flowing into the first sample injection port (161) and the second sample injection port (162) are mixed through the Y-shaped micro-channel (160) and then discharged from the outflow port (163); the first L-shaped micro-channel (130) communicates the first sample loading chamber (120) with the first sample injection port (161) of the Y-shaped micro-channel (160); the second L-shaped micro-channel (150) communicates the second sample loading chamber (140) with the second sample injection port (162) of the Y-shaped micro-channel (160); and the outflow port (163) of the Y-shaped micro-channel (160) is communicated with the reaction detection cavity (170).

2. The micro-fluidic chip for blood detection according to claim 1, wherein the first L-shaped micro-channel (130) and the second L-shaped micro-channel (150) are both composed of a lower-layer micro-channel and a vertical micro-channel which are sequentially communicated, and the lower-layer micro-channel is arranged vertical to the vertical micro-channel; an inlet of the lower-layer micro-channel in the first L-shaped micro-channel (130) is communicated with a bottom portion on one side of the first sample loading chamber (120) far away from the center position of the chip body (1); and an inlet of the lower-layer micro-channel in the second L-shaped micro-channel (150) is communicated with a bottom portion on one side of the second sample loading chamber (140) far away from the center position of the chip body (1);
the first sample injection port (161), the second sample injection port (162) and the outflow port (163) of the Y-shaped micro-channel (160) are all located at an upper portion of the chip body (1); and the first sample injection port (161) of the Y-shaped micro-channel (160) is communicated with an outlet of the vertical micro-channel in the first L-shaped micro-channel (130), and the second sample injection port (162) of the Y-shaped micro-channel (160) is communicated with an outlet of the vertical micro-channel in the second L-shaped micro-channel (150); and
the Y-shaped micro-channel (160) comprises two upper-layer inlet micro-channels and one upper-layer mixing micro-channel; an inlet of one upper-layer inlet micro-channel is the first sample injection port (161) of the Y-shaped micro-channel (160), an inlet of the other upper-layer inlet micro-channel is the second sample injection port (162) of the Y-shaped micro-channel (160), outlets of the two upper-layer inlet micro-channels intersect at an inlet of the upper-layer mixing micro-channel, and an outlet of the upper-layer mixing micro-channel is the outflow port (163) of the Y-shaped micro-channel (160); and each upper-layer inlet micro-channel is arranged vertical to the corresponding vertical micro-channel.

3. The micro-fluidic chip for blood detection according to claim 2, wherein the trace blood separation tank (110) is arranged along a radial direction of the chip; the trace blood separation tank (110) comprises a plasma extraction groove (111) and a straight pipe groove (112) communicated with the plasma extraction groove (111); the straight pipe groove (112) is located at one end of the trace blood separation tank (110) far away from the center position of the chip, and the plasma extraction groove (111) is located at the other end of the trace blood separation tank (110) close to the center position of the chip; and under an action of a centrifugal force, all the erythrocytes obtained after separation of the trace whole blood sample sediment at one end of the straight pipe groove (112) far away from the center position of the chip.

4. The micro-fluidic chip for blood detection according to claim 3, wherein a surface of the straight pipe groove (112) is provided with a scale mark for interpreting a hematocrit value; and a volume of the trace blood separation tank (110) is 50 ul to 100 ul.

5. The micro-fluidic chip for blood detection according to claim 1, wherein a quantity of the reaction test units is six; and the chip further comprises plasma multiple-proportion dilution pretreatment tanks arranged in one-to-one correspondence with the reaction test units.

6. The micro-fluidic chip for blood detection according to claim 1, wherein the reaction detection cavity (170) comprises a cylindrical chamber and a tapered chamber with a gradually reduced diameter, and a large-diameter end of the tapered chamber is communicated with a bottom end of the cylindrical chamber.

7. The micro-fluidic chip for blood detection according to claim 1, wherein the micro-fluidic chip is used as a detection method that comprises the following steps of:
allowing a first micro-fluid of the first sample loading chamber (120) to flow into the first sample injection port (161) of the Y-shaped micro-channel (160) through the first L-shaped micro-channel (130); and allowing a second micro-fluid of the second sample loading chamber (140) to flow into the second sample injection port (162) of the Y-shaped micro-channel (160) through the second L-shaped micro-channel (150); and
mixing the first micro-fluid and the second micro-fluid in the Y-shaped micro-channel (160) to form a first mixture, and allowing the first mixture to flow into the reaction detection cavity (170) through the outflow port (163) of the Y-shaped micro-channel (160).

8. A method for carrying out ABO blood group reverse typing detection by using the micro-fluidic chip for blood detection according to claim 3, wherein a quantity of the reaction test units is three; the erythrocyte reagents received by the second sample adding cavities (140) of the reaction test units are respectively an A-group erythrocyte reagent, a B-group erythrocyte reagent and an O-group erythrocyte reagent; and the detection method comprises the following steps:
first step: receiving a to-be-detected trace whole blood sample by the trace blood separation tank (110);
second step: under the action of the centrifugal force, separating erythrocytes and plasma in the to-be-detected trace whole blood sample, allowing the separated erythrocytes to sediment at one end of the straight pipe groove (112) far away from the center position of the chip, accommodating a first part of separated plasma in the plasma extraction groove (111), and accommodating a second part of separated plasma in one side of the straight pipe groove (112) close to the center position of the chip;
third step: after centrifugation, sucking the plasma in the plasma extraction groove (111) and transferring the plasma to the first sample loading chamber (120) of each reaction test unit; and receiving the corresponding erythrocyte reagent by the second sample loading chamber (140) of each reaction test unit;
fourth step: under the action of the centrifugal force, allowing the plasma of the first sample loading chamber (120) to flow into the first sample injection port (161) of the Y-shaped micro-channel (160) through the first L-shaped micro-channel (130); allowing the erythrocyte reagent of the second sample loading chamber (140) to flow into the second sample injection port (162) of the Y-shaped micro-channel (160) through the second L-shaped micro-channel (150); and mixing the plasma and the erythrocyte reagent in the Y-shaped micro-channel (160) to form a first mixture, and allowing the first mixture to flow into the reaction detection cavity (170) through the outflow port (163) of the Y-shaped micro-channel (160) to fully react in the reaction detection cavity (170); and
fifth step: allowing the chip to stand, and interpreting to obtain a detection result;
wherein, in a case that a blood group antibody in the to-be-detected plasma is subjected to an immune agglutination reaction with a blood group antigen in the erythrocyte reagent, an erythrocyte clot is formed; under the action of the centrifugal force, the erythrocyte clot sediments in a direction far away from the center position of the chip; and when the chip is stationary, the erythrocyte clot remains adhered to an inner side wall of the reaction detection cavity (170) for a certain period of time; and in a case that the blood group antibody in the to-be-detected plasma is not subjected to the immune agglutination reaction with the blood group antigen in the erythrocyte reagent, under the action of the centrifugal force, the erythrocytes without the immune agglutination reaction sediment in the direction far away from the center position of the chip; and when the chip is stationary, the erythrocytes without the immune agglutination reaction naturally collapse and sediment due to an action of gravity.

9. A method for detecting a titer of a blood group IgG antibody of a HDN pregnant woman by using the micro-fluidic chip for blood detection according to claim 3, wherein a quantity of the reaction test units is six; each reaction test unit is also correspondingly provided with an independent plasma multiple-proportion dilution pretreatment tank for forming a series of multiple-proportion dilutions; the second sample loading chamber (140) of each reaction test unit receives the same erythrocyte reagent, and the same erythrocyte reagent is an A-group erythrocyte reagent, a B-group erythrocyte reagent or an O-group RhD-positive erythrocyte reagent; and the detection method comprises the following steps:
first step: receiving a to-be-detected trace whole blood sample by the trace blood separation tank (110);
second step: under the action of the centrifugal force, separating erythrocytes and plasma in the to-be-detected trace whole blood sample, allowing the separated erythrocytes to sediment at one end of the straight pipe groove (112) far away from the center position of the chip, accommodating a first part of separated plasma in the plasma extraction groove (111), and accommodating a second part of separated plasma in one side of the straight pipe groove (112) close to the center position of the chip;
third step: after centrifugation, sucking the plasma in the plasma extraction groove (111) and transferring the plasma to each plasma multiple-proportion dilution pretreatment tank sequentially;
fourth step: pretreating the plasma in each plasma multiple-proportion dilution pretreatment tank with a diluent containing dithiothreitol (DTT) or 2-mercaptoethanol (2-ME), and allowing the plasma to stand for reaction for 15 minutes to 30 minutes to destroy an activity of an IgM antibody; and after standing reaction, adding a sample diluent to complete multiple-proportion dilution, so as to obtain plasma subjected to multiple-proportion dilution;
fifth step: sucking the plasma subjected to multiple-proportion dilution in each plasma multiple-proportion dilution pretreatment tank and transferring the plasma subjected to multiple-proportion dilution to the first sample loading chamber (120) of the corresponding reaction test unit; and receiving the erythrocyte reagent by the second sample loading chamber (140) of each reaction test unit;
sixth step: under the action of the centrifugal force, in each reaction test unit, allowing the plasma subjected to multiple-proportion dilution of the first sample loading chamber (120) to flow into the first sample injection port (161) of the Y-shaped micro-channel (160) through the first L-shaped micro-channel (130); allowing the erythrocyte reagent of the second sample loading chamber (140) to flow into the second sample injection port (162) of the Y-shaped micro-channel (160) through the second L-shaped micro-channel (150); and mixing the plasma subjected to multiple-proportion dilution and the erythrocyte reagent in the Y-shaped micro-channel (160) to form a first mixture, and allowing the first mixture to flow into the reaction detection cavity (170) through the outflow port (163) of the Y-shaped micro-channel (160) to fully react in the reaction detection cavity (170); and
seventh step: allowing the chip to stand, and interpreting to obtain a detection result;
wherein, in a case that a blood group antibody in the to-be-detected plasma is subjected to an immune agglutination reaction with a blood group antigen in the erythrocyte reagent, an erythrocyte clot is formed; under the action of the centrifugal force, the erythrocyte clot sediments in a direction far away from the center position of the chip; and when the chip is stationary, the erythrocyte clot remains adhered to an inner side wall of the reaction detection cavity (170) for a certain period of time; and
in a case that the blood group antibody in the to-be-detected plasma is not subjected to the immune agglutination reaction with the blood group antigen in the erythrocyte reagent, under the action of the centrifugal force, the erythrocytes without the immune agglutination reaction sediment in the direction far away from the center position of the chip; when the chip is stationary, the erythrocytes without the immune agglutination reaction naturally collapse and sediment due to an action of gravity; and a reciprocal value of a dilution multiple of a plasma sample subjected to highest-multiple-proportion dilution without the immune agglutination reaction is taken as a titer of a blood group IgG antibody against a specific blood group antigen.

10. The method according to claim 9, wherein an anti-human globulin polyclonal antibody lyophilized bead is preset in the reaction detection cavity (170) of each reaction test unit.
